(19) United States
(12) Patent Application Publication
(10) Pub. No.: US 2009/0004182 A1
(43) Pub. Date: Jan. 1, 2009

(54) Methods to Treat or Prevent Viral-Associated Lymphoproliferative Disorders

(75) Inventors: Robert A. Baiocchi, Columbus, OH (US); Michael A. Caligiuri, Columbus, OH (US); Anne M. Van Buskirk, Westerville, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 11/577,111

(22) PCT Filed: Oct. 12, 2005
(86) PCT No.: PCT/US2005/036613

§ 371 Date: May 27, 2008

Related U.S. Application Data
(60) Provisional application No. 60/618,458, filed on Oct. 13, 2004.

(51) Int. Cl. A61K 39/395; C12Q 1/68; C12Q 1/02

(52) U.S. Cl. 424/133.1; 424/158.1; 424/172.1; 435/6; 435/29

(57) ABSTRACT

The disclosure relates to methods to prevent, treat, or slow the progression viral-associated lymphoproliferative disorders, EBV-associated lymphoproliferative disorders, and post-transplant lymphoproliferative disorders. In the methods, a TGF-β antagonist, e.g., an anti-TGF-β antibody is administered to a subject. Methods for treating viral-associated lymphoproliferative disorders and for enhancing T-cell responsiveness to a viral-associated lymphoproliferative disorder by administering a TGF-β antagonist are also described.

This application claims priority to U.S. Provisional Application No. 60/618,458, filed Oct. 13, 2004, the entire disclosure of which is incorporated herein by reference.

### BACKGROUND

Proliferative disorders, including lymphoproliferative disorders (LPDs), are frequently associated with immunosuppression. For example, immunosuppressive therapy following organ or tissue transplantation is associated with certain neoplasms, and many LPDs develop in the background of immune deficiencies, including viral infection (reviewed in Brusamolino et al., Haematologica 74:605-622 (1989)).

Post-transplant lymphoproliferative disorder (PTLD) is a devastating complication of solid organ and stem cell transplantation that can have 70-80% mortality (Paya et al., Transplantation 68:1517-1525 (1999)). PTLD is often associated with Epstein-Barr virus (EBV), a herpes virus that establishes latent infection in a majority of healthy adults. The incidence of PTLD varies according to the organ transplanted, as well as the intensity and duration of immunosuppression. In renal transplant recipients PTLD occurs in 1-2% of patients, but the incidence is as high as 20% in bone marrow and in lung transplant recipients (Paya et al., supra). Children and transplant recipients without previously established anti-EBV immunity are among those at greatest risk for development of a PTLD. There is no accepted standard of therapy for PTLD, and the progression of the disease in patients is often not responsive to currently available therapies. However, it is believed that cytotoxic T-lymphocyte (CTL) activity is involved in prevention and recovery from PTLD.

It is thought that immunosuppression inhibits the EBV-specific cellular immunity that normally prevents the progression of EBV-driven transformation of latently infected cells. Reduction of immunosuppression is effective in treating some, but not all PTLD patients (Paya et al., supra), but such therapy increases the likelihood of developing acute rejection episodes that can result in graft loss and other serious complications. Anti-viral, cellular, and monoclonal antibody therapies directed to CD-20 protein may be indicated for treatment of some PTLD patients; however, none are completely satisfactory (Liu et al., Recent Results Cancer Res. 159:123-133 (2002); Zilz et al., J. Heart Lung Transplant 20:770-772 (2001)).

In preliminary clinical observations of nine PTLD patients, a particular IFN-γ cytokine genotype that is associated with low IFN-γ production was shown to be prevalent in renal transplant recipients who develop PTLD (VanBuskirk et al., Transplant. Proc. 33:1834 (2001)). IFN-γ is a critical regulatory cytokine in cellular immunity that is important for immune surveillance. One polymorphism in the IFN-γ gene is a single nucleotide polymorphism at position +874 containing either a thymidine (T) or an adenosine (A). The presence of the thymidine at +874 correlates with microsatellite repeats associated with high cytokine production and creates an NF-kB binding site (Pravica et al., Biochem. Soc. Trans. 25:176S (1997); Pravica et al., Eur. J. Immunogenetics 26:1-3 (1999); Pravica et al., Hum. Immunol. 61:863-866 (2000)). The T/T genotype is often referred to as a “high producer” and A/A genotype as “low producer” (Pravica et al., Hum. Immunol. 61:863-866 (2000)). In the clinical study, the low producing, A/A IFN-γ genotype was present in 80% of the nine PTLD patients, compared to 27% of 135 non-PTLD renal transplant patients (VanBuskirk et al., supra), and the polymorphism was identified as a possible risk factor for PTLD development.

Transforming growth factor-β (TGF-β) is antagonistic to IFN-γ and has been implicated in EBV activation, replication, and increased transformation (Schuster et al., FEBS Lett. 284:82-86 (1991); diRenzo et al., Int. J. Cancer 57:914-919 (1994); Liang et al., J. Biol. Chem. 277:23345-23357 (2002); Fahmi et al., J. Virol. 74:5810-5818 (2000)). TGF-β is also a ubiquitous, pluripotent cytokine that suppresses multiple T cell and antigen presenting cell (APC) functions, including T cell effector function, and may otherwise inhibit immune surveillance ((see Letterio et al., Annu. Rev. Immunol. 16:137-161 (1998); Gold, Crit. Rev. Oncog. 10:303-360 (1999); Altiok et al., Immunol. Lett. 40:111-115 (1994)). The antagonistic and counter-regulatory activities of TGF-β and IFN-γ are reviewed in Strober et al., Immunol. Today 18:61-64 (1997), and studies have shown that IFN-γ can inhibit TGF-β activity, and vice versa.

As current therapies are not optimal, there is a need for methods and compositions for treating or preventing viral-associated LPDs, including PTLDs. There is also a need for methods of treating lymphoproliferative disorders associated with low IFN-γ levels, and/or insufficient T cell responsiveness. Further means to identify patients that are candidates for treatment, including candidates for receiving specific therapies, are needed.

### SUMMARY OF THE INVENTION

The present invention relates to the discovery that inhibition of TGF-β activity, for example by administration of a TGF-β antagonist, prevents, treats, or slows the progression viral-associated lymphoproliferative disorders (LPD), including post-transplant lymphoproliferative disorder (PTLD). Administration of a TGF-β antagonist results in protection from LPD and an expansion of human CD8+ cells. Additionally, expansion of CD8+ T cells and activation of CD8+ T cells correlate with inhibition of TGF-β activity and inhibition of LPD.

The present invention provides methods for treating, preventing, and reducing the risk of occurrence of viral-associated LPDs, including EBV-associated LPDs and PTLD. The invention further provides methods for enhancing T cell responsiveness to viral infection, such as, e.g., a herpes virus, HHV-8, cytomegalovirus, Epstein-Barr virus (EBV), C-type retrovirus, human T-lymphotropic virus type 1 (C-type retrovirus), and/or human immunodeficiency virus (HIV, HIV-1, HIV-2), for example. The disclosed methods include administering to a mammalian subject at risk for, susceptible to, or afflicted with, an LPD, therapeutically effective amounts of a TGF-β antagonist. The populations treated by the methods of the invention include but are not limited to subjects suffering from, or at risk for the development of an LPD, including, e.g., subjects with immune deficiency or who have been treated to induce immunosuppression. In certain embodiments, methods for treating viral-associated disorders in individuals with low IFN-γ levels are provided.

The invention further provides methods for assessing the presence of one or more risk factors for the development of a viral-associated LPD, or its progression or responsiveness to treatment, and administering a TGF-β antagonist to subject having the risk factor. For example, methods comprising assessing or measuring IFN-γ levels or IFN-γ genotype, and treating a subject with low IFN-γ levels or with the A/T or A/A+874 genotype are provided herein.

Methods of administration and compositions used in the methods of the inventions are provided. In the disclosed methods, TGF-β antagonists include, but are not limited to, antibodies directed against one or more isoforms of TGF-β; antibodies directed against TGF-β receptors; soluble TGF-β receptors and fragments thereof; and TGF-β inhibiting sugars and proteoglycans, and small molecule inhibitors of TGF-β.

In certain embodiments, the TGF-β antagonist is a monoclonal antibody or a fragment thereof that blocks TGF-β binding to its receptor. Nonlimiting illustrative embodiments include a non-human monoclonal anti-TGF-β antibody, e.g., mouse monoclonal antibody 1D11 (also known as 1D11.16, ATCC Deposit Designation No. HB 9849), a derivative thereof (e.g., a humanized antibody) and a fully human monoclonal anti-TGF-β1 antibody (e.g., CAT192 described in WO 00/66631) or a derivative thereof.

The foregoing summary and the following description are not restrictive of the invention as claimed.

### BRIEF DESCRIPTION OF THE DRAWINGS

FIG. 1A shows the effect of TGF-β in a cytolysis assay comparing peripheral blood lymphocytes (PBL) from individuals with the A/A, A/T, or T/T IFN-γ genotype. FIG. 1B shows effect of TGF-β on the ability of CTL to prevent matched LCL growth is inhibited by CTL re-stimulation in the presence of TGF-β.

FIG. 2 shows that treatment with anti-TGF-β antibody prevents death from LPD in a human PBL-severe combined immunodeficiency (hu PBL-SCID) mouse model of lymphoproliferative disease.

FIG. 3A shows that anti-TGF-β antibody neutralizes TGF-β in vivo. FIG. 3B demonstrates that anti-TGF-β antibody reduces the incidence of LPD in a dose dependent manner in the hu PBL-SCID model.

FIG. 4A shows a flow cytometric analysis of tumors in anti-TGF-β antibody and control treated hu PBL-SCID mice, and FIG. 4B shows cytometric analysis of spleens from anti-TGF-β antibody and control treated hu PBL-SCID mice. These data demonstrate while that tumors and spleens from control IgG-treated mice contained human B cells and very few CD8+ T cells, but large numbers of CD8+ T cells are present in tumors and spleens of anti-TGF-β treated hu PBL-SCID mice.

### DETAILED DESCRIPTION

The present invention is based, in part, on the discovery and demonstration that inhibition or neutralization of TGF-β with a TGF-β antagonist, such as an anti-TGF-β antibody, reduces the occurrence and progression of a viral-associated LPD in a mammalian subject. The data show that use of a TGF-β antagonist prevents or inhibits the progression of tumor development associated with low IFN-γ levels in a subject treated therewith. These data also show that administration of a TGF-β antagonist reverses TGF-β inhibition of CTL restimulation and expansion. Neutralization of TGF-β in a mouse model of LPD results in expansion of CD8+ cells, and reduces LPD development. Additionally, the data indicate that IFN-γ genotype provides valuable information in identifying transplant recipients at greater risk for PTLD, for example, and in developing preventative and curative strategies. Accordingly, the present invention provides methods for treating, preventing, and reducing the risk of occurrence of a viral-associated disorder and an LPD, such as a viral-associated LPD, EBV-associated LPD and/or post-transplant lymphoproliferative disorder, in mammals.

In order that the present invention may be more readily understood, certain terms are first defined. Additional definitions are set forth throughout the detailed description.

The term “antibody,” as used herein, refers to an immunoglobulin or a part thereof, and encompasses any polypeptide comprising an antigen-binding site regardless of the source, method of production, and other characteristics. The term includes, but is not limited to, polyclonal, monoclonal, monospecific, polyspecific, humanized, human, single-chain, chimeric, synthetic, recombinant, hybrid, mutated, and CDR-grafted antibodies. As will be recognized by those of skill in the art, any of such molecules, e.g., a “human” antibody, may be engineered (for example “germlined”) to decrease its immunogenicity, increase its affinity, alter its specificity, or for other purposes. The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is responsible for specific interactions with the antigen-binding domain of an antibody. An antigen-binding domain may comprise an antibody light chain variable region (V_{L}) and an antibody heavy chain variable region (V_{H}) or portions thereof. An antigen-binding domain may be provided by one or more antibody variable domains (e.g., a so-called Fd antibody fragment consisting of a V_{H} domain or a so-called Fv antibody fragment consisting of a V_{H} domain and a V_{L} domain). The term “anti-TGF-β antibody,” or “antibody against at least one isoform of TGF-β,” refers to any antibody that specifically binds to at least one epitope of TGF-β. The terms “TGF-β receptor antibody” and “antibody against a TGF-β receptor” refer to any antibody that specifically binds to at least one epitope of a TGF-β receptor (e.g., type I, type II, or type III).

The terms “therapeutic compound” as used herein, refer to any compound capable of modulating or inhibiting a TGF-β by affecting a biological activity of TGF-β, either directly or indirectly.

The terms “inhibit,” “neutralize,” “antagonize,” and their cognates refer to the ability of a compound to act as an antagonist of a certain reaction or biological activity. The decrease in the amount or the biological activity is preferably at least about 10%, 20%, 30%, 40%, 50%, 60%, 70%, 80%, 90%, or more. The terms refer to a decrease in the relative amount or activity of at least one protein that is responsible for the biological activity of interest (e.g., TGF-β and TGF-β receptor). Additionally, the terms refer to a relative decrease in a biological activity of TGF-β or TGF-β receptor, for example, as measured in an assay (e.g., T cell cytotoxicity, activation, or proliferation assays), or as described herein.

As used herein, “TGF-β antagonist” and its cognates such as “inhibitor,” “neutralizing agent,” and “downregulating agent” refer to a compound (or its property, as appropriate), which acts as an antagonist of a biological activity of TGF-β. A TGF-β antagonist may, for example, bind to and neutralize the activity of TGF-β; decrease TGF-β expression levels; affect stability or conversion of the precursor molecule to the active, mature form; interfere with the binding of TGF-β to one or more receptors; or it may interfere with intracellular signaling of a TGF-β receptor. The term “direct TGF-β antagonist” generally refers to any compound that directly downregulates the biological activity of TGF-β. A molecule “directly downregulates” the biological activity of TGF-β if it downregulates the activity by interacting with a TGF-β gene, a TGF-β transcript, a TGF-β polypeptide, a TGF-β ligand, or a TGF-β receptor. Methods for assessing neutralizing biological activity of TGF-β antagonists are known in the art and examples are described infra.

The terms “lymphoproliferative disorder,” “LPD” and their cognates refer to a disorder in which lymphocytes, white blood cells produced in the lymphatic tissue (the lymph nodes, spleen, thymus, for example), are over-produced or act abnormally. An LPD involves aberrant proliferation of lymphocytes or lymphatic tissues, i.e. a “viral-associated lymphoproliferative disorder,” or “post-transplant lymphoproliferative disorder,” for example. Lymphoid cells include thymus derived lymphocytes (T cells); bone marrow-derived lymphocytes (B cells), and natural killer (NK cells), for example. Lymphocytes progress through a number of different stages, including proliferation, activation, and maturation, and lymphoma or aberrant proliferation can develop at each stage. Disorders may be malignant neoplasms (and may be classified as aggressive or indolent, or as low, intermediate or high-grade), including those associated with IFN-γ, or the disorders may involve non-malignant aberrant expansion of lymphoid cells. LPDs include any monoclonal or polyclonal LPD that is not resolving without treatment and/or that involves excessive cellular proliferation, such as an expanding, monoclonal, polyclonal or oligoclonal, lymphoid neoplasm. Cellular proliferation may be more rapid than normal and may continue after the stimuli that initiated the new growth cease. A neoplasm will show partial or complete lack of structural organization and functional coordination with the normal tissue, and may form a distinct mass of tissue that may be either benign (benign tumor) or malignant (cancer). Methods to detect aberrant proliferation, function, or structure of a lymphatic (or other) cell or tissue may be used to diagnose, monitor the progression of, or assay the efficacy of a therapeutic agent for a viral-associated LPD, such as PTLD. In certain embodiments, LPDs do not include cancers. In other embodiments, viral-associated LPDs do not include cancers.

Such diseases or disorders include, but are not limited to, T-cell lymphoproliferative disease, lymphoma, Hodgkin's lymphoma, non-Hodgkin's lymphoma, aggressive large-cell lymphoma, post-transplant lymphoproliferative disorder, AIDS-associated lymphoma, Burkitt's lymphoma, Karposi sarcoma, and Epstein-Barr virus-associated lymphoma. “Post-transplant lymphoproliferative disorder” or “PTLD” refers to varied hyperplastic and/or neoplastic disorders that are associated with organ, tissue, or stem cell transplantation and concomitant immune suppressive therapy. PTLD includes disorders ranging from lymphocyte hyperplasia, such as reactive polyclonal B-cell hyperplasia, to polyclonal or monoclonal B-cell lymphoma, for example. Examples of aggressive non-Hodgkin's lymphomas include, but are not limited to, diffuse large cell lymphoma, Burkitt's lymphoma, lymphoblastic lymphoma, central nervous system lymphoma, adult T-cell leukemia/lymphoma (HTLV-1+), mantle-cell lymphoma, post-transplant lymphoproliferative disorder, AIDS-associated lymphoma, true histiocytic lymphoma, primary effusion lymphoma, and aggressive NK-cell leukemia. Examples of indolent non-Hodgkin's lymphomas include, but are not limited to, follicular lymphoma, diffuse small lymphocytic lymphoma/chronic lymphocytic leukemia, lymphoplastic lymphoma, Waldenstrom's macroglobulinemia, MALT (extranodal marginal zone B-cell lymphoma), monocytoid B-cell lymphoma (nodal marginal zone B-cell lymphoma), splenic lymphoma with villous lymphocytes (splenic marginal zone lymphoma), hairy-cell leukemia, and mycosis fungoides/Sezary syndrome.

“Viral-associated” proliferative disorders refer to an LPD caused by or correlated with a virus. Viral-associated LPD may be caused by or associated with, e.g., a herpes virus, HHV-8, cytomegalovirus, Epstein-Barr virus (EBV), C-type retrovirus, human T-lymphotropic virus type 1 (C-type retrovirus), and/or human immunodeficiency virus (HIV, HIV-1, HIV-2), for example. HIV or AIDS-associated cancers include HIV-associated LPDs, and examples are Karposi sarcoma, non-Hodgkin's lymphoma, central nervous system (CNS) lymphoma, adult T-cell leukemia/lymphoma (HTLV-1+), and AIDS-associated lymphoma. “EBV-associated” disorders include mononucleosis, nasopharyngeal carcinoma, invasive breast cancer, gastric carcinomas, and EBV-associated LPDs, for example. “EBV-associated LPDs” include, but are not limited to, primary CNS lymphomas, PTLD, Burkitt's lymphoma, T-cell lymphoma, X-linked LPDs, Chédiak-Higashi syndrome, Hodgkin's lymphoma, and non-Hodgkin's lymphoma. Approximately 40% of refractory non-Hodgkin's lymphoma, e.g., mantle cell lymphoma, diffuse large B cell lymphomas, and NK/T cell lymphomas, for example, is associated with EBV. X-linked LPD often involves a T-cell-mediated response to EBV viral infection. Immune deficiency such as in AIDs patients, organ transplant recipients, and genetic immune disorders may allow latent EBV to reactivate, causing proliferation of abnormal lymphocytes and the potential to develop an EBV-associated LPD, for example. Methods to detect the presence of virus or viral infection in an aberrant cell, such as a cell involved in an LPD, are known in the art. Viral nucleic acid or polypeptides may be detected in a cell, tissue, or organism such as an aberrant cell, for example. Also, methods to detect immune response specific for a virus are known. A delayed type-hypersensitivity (DTH) assay, such as a trans-vivo DTH assay may be used to detect regulatory T cells, for example. In such an assay, human or other mammalian peripheral blood mononuclear cells (PBMC) are mixed with a carrier control with and without viral antigen, for example, and injected into a heterologous naïve recipient, such as the pinnae or footpad of naïve mice. If the donor of the PBMC had previously been sensitized to the challenge antigen, DTH-like swelling responses are observed.

A subject “at risk” for an LPD associated with low IFN-γ, or a viral-associated LPD with or without being associated with low IFN-γ levels, is a subject with one or more risk factors that increase the likelihood of developing the disorder. One of the factors that puts a subject at risk for developing a viral-associated LPD, or a PTLD, is if he or she is homozygous or heterozygous for a low producer IFN-γ genotype, such as an A/A or A/T genotype at position +874 of the IFN-γ gene. A subject at risk for an LPD associated with low IFN-γ levels or viral-associated LPD may have one or more other risk factors, including: immune deficiency; immunosuppressive therapy; organ, tissue, or cell transplantation (including stem cell transplantation); EBV sero-negative status prior to transplantation; EBV reactivation; reactivation of a latent virus; primary EBV or other viral infection in an immune deficient patient; age of the subject (i.e., child or adult); and the type and duration of immunosuppressive therapy administered to prevent graft rejection, among others. A subject at risk may be identified, for example, by evaluating viral loads in blood and tissues (for example looking for increased viral load after transplant), or by testing for increased numbers of leukocytes, B cells, or total serum IgM. EBV (or other virus) may be detected by Southern blot hybridization or by polymerase chain reaction (PCR), including quantitative or semiquantitative PCR, or by positive viral serology (anti-viral capsid antigen IgG (EBV serology)) in the blood, serum, or tissue of a subject, as appropriate.

“Immune deficiency” may be inherited, acquired, or iatrogenic (induced by diagnostic, medical therapy, or surgical procedures). Examples of inherited immune deficiency include, for example, severe combined immune deficiency, autoimmune diseases, X-linked immune deficiencies, X-linked agammaglobulinemia, common variable immune deficiency, Chédiak-Higashi syndrome, Wiskott-Aldrich syndrome, or Ataxia telangiectasia. Acquired immunodeficiency may be caused by disease or infection such as with human immunodeficiency virus (HIV). Iatrogenic immune deficiencies include those caused by immunosuppressive therapy, including therapy concomitant to transplantation of organ or tissue. Immunosuppressive therapy refers to administration of a compound or composition that induces immunosuppression, i.e., it prevents or interferes with the development of an immunologic response. Therapeutic immunosuppression may involve administration of cyclosporine, azathioprine, and/or prednisolone, as well as other immunosuppressive agents, including those listed elsewhere in this description.

The terms “treatment,” “therapeutic method,” and their cognates refer to treatment or prophylactic/preventative measures. Those in need of treatment may include individuals already having a particular medical disorder as well as those who may ultimately acquire the disorder. The need for treatment may be assessed, for example, by the presence of one or more risk factors associated with the development of a disorder, the presence or progression of a disorder, or likely receptiveness to treatment of a subject having the disorder. Treatment may include slowing or reversing the progression of a disorder.

The terms “therapeutically effective dose,” or “therapeutically effective amount,” refer to that amount of a compound that results in prevention or delay of onset or amelioration of symptoms of an LPD, viral-associated LPD, EBV-associated LPD, and/or post-transplant LPD in a subject or an attainment of a desired biological outcome, such as reduced aberrant proliferation. The effective amount can be determined by methods well known in the art and as described in subsequent sections of this description.

The terms “specific interaction,” “specifically binds,” or their cognates, mean that two or more molecules form a complex that is relatively stable under physiologic conditions. Specific binding is characterized by a high affinity and a low to moderate capacity. Nonspecific binding usually has a low affinity with a moderate to high capacity. Typically, the binding is considered specific when the affinity constant Kₐ is higher than 10⁶ M⁻¹, or preferably higher than 10⁸ M⁻¹. If necessary, nonspecific binding can be reduced without substantially affecting specific binding by varying the binding conditions. Such conditions are known in the art, and a skilled artisan using routine techniques can select appropriate conditions. The conditions are usually defined in terms of concentration of binding proteins, ionic strength of the solution, temperature, time allowed for binding, concentration of unrelated molecules (e.g., serum albumin, milk casein), etc.

The phrase “substantially identical” means that a relevant amino acid sequence is at least 70%, 75%, 80%, 85%, 90%, 95%, 97%, 98%, 99%, or 100% identical to a given sequence. By way of example, such sequences may be variants derived from various species, or they may be derived from the given sequence by truncation, deletion, amino acid substitution or addition. Mutants, fragments, or derivatives of a TGF-β antagonist, for example, may have substantially identical amino acid or nucleic acid sequences as compared to the TGF-β antagonist, and retain the ability to directly inhibit the biological activity of TGF-β. Percent identity between two amino acid sequences may be determined by standard alignment algorithms such as, for example, Basic Local Alignment Tool (BLAST) described in Altschul et al., J. Mol. Biol., 215:403-410 (1990), the algorithm of Needleman et al., J. Mol. Biol., 48:444-453 (1970), or the algorithm of Meyers et al., Comput. Appl. Biosci. 4:11-17 (1988). Such algorithms are incorporated into the BLASTN, BLASTP, and “BLAST 2 Sequences” programs (see www.ncbi.nlm.nih.gov/BLAST). When utilizing such programs, the default parameters can be used. For example, for nucleotide sequences the following settings can be used for “BLAST 2 Sequences”: program BLASTN, reward for match 2, penalty for mismatch −2, open gap and extension gap penalties 5 and 2 respectively, gap x_dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x_dropoff 50, expect 10, word size 3, filter ON. The amino acid and nucleic acid sequences of this application, including those incorporated by reference, may include homologous, variant, or substantially identical sequences.

As used herein, “TGF-β,” unless otherwise specifically indicated, refers to any one or more isoforms of TGF-β. Likewise, the term “TGF-β receptor,” unless otherwise indicated, refers to any receptor that binds at least one TGF-β isoform. Currently, there are 5 known isoforms of TGF-β (TGF-β1-β5), all of which are homologous among each other (60-80% identity), form homodimers of about 25 kDa, and act upon common TGF-β receptors (TβR-I, TβR-II, TβR-IIB, and TβR-III). TGF-β1, TGF-β2, and TGF-β3 are found in mammals. The structural and functional aspects of TGF-β, as well as TGF-β receptors, are well known in the art (see, for example, Cytokine Reference, eds. Oppenheim et al., Academic Press, San Diego, Calif., 2001). TGF-β is remarkably conserved among species. For example, the amino acid sequences of rat and human mature TGF-β1s are nearly identical. Thus, antagonists of TGF-β are expected to have high species cross-reactivity.

### TGF-β Antagonists

TGF-β is a disulfide linked dimer that is synthesized as a preproprotein of about 400 amino acids (aa) which is cleaved prior to secretion to produce mature TGF-β. The N-terminal cleavage fragment, known as the “latency-associated peptide” (LAP), may remain noncovalently bound to the dimer, thereby inactivating TGF-β. TGF-β, isolated in vivo, is found predominantly in this inactive, “latent” form associated with LAP. Latent TGF-β complex may be activated in several ways, for example, by binding to cell surface receptors called the cation-independent mannose-6-phosphate/insulin-like growth factor II receptor. Binding occurs through mannose-6-phosphate residues attached at glycosylation sites within LAP. Upon binding to the receptor, TGF-β is released in its mature form. Mature, active TGF-β is then free to bind to its receptor and exert its biological functions. The major TGF-β3-binding domain in the type II TGF-β receptor has been mapped to a 19 amino acid sequence (Demetriou et al., J. Biol. Chem., 271:12755 (1996)).

Examples of TGF-β antagonists that may be used in the methods of the present invention include, but are not limited to: monoclonal and polyclonal antibodies directed against one or more isoforms of TGF-β (U.S. Pat. No. 5,571,714; WO 97/13844; WO 00/66631; dominant negative and soluble TGF-β receptors or antibodies directed against TGF-β receptors (Flavell et al., Nat. Rev. Immunol. 2:46-53 (2002); U.S. Pat. No. 5,693,607; U.S. Pat. No. 6,001,969; U.S. Pat. No. 6,008,011; U.S. Pat. No. 6,010,872; WO 92/00330; WO 93/09228; WO 95/10610; and WO 98/48024); LAP (WO 91/08291); LAP-associated TGF-β (WO 94/09812); TGF-β-binding glycoproteins/proteoglycans such as fetuin (U.S. Pat. No. 5,821,227); decorin, biglycan, fibromodulin, lumican, and endoglin (U.S. Pat. No. 5,583,103; U.S. Pat. No. 5,654,270; U.S. Pat. No. 5,705,609; U.S. Pat. No. 5,726,149; U.S. Pat. No. 5,824,655; U.S. Pat. No. 5,830,847; U.S. Pat. No. 6,015,693; WO 91/04748; WO 91/10727; WO 93/09800; and WO 94/10187); TGF-β accessory receptors, including receptors that directly bind TGF-β1 such as r150 protein, its soluble forms, derivatives or precursors (U.S. Patent Pub. No. 20040191860); mannose-6-phosphate or mannose-1-phosphate (U.S. Pat. No. 5,520,926); prolactin (WO 97/40848); insulin-like growth factor 11 (WO 98/17304); extracts of plants, fungi and bacteria (EU 813875; JP 8119984; and U.S. Pat. No. 5,693,610); antisense oligonucleotides (U.S. Pat. No. 5,683,988; U.S. Pat. No. 5,772,995; U.S. Pat. No. 5,821,234; U.S. Pat. No. 5,869,462; and WO 94/25588); small molecule inhibitors, such as serine/threonine kinase inhibitors (WO 04/21989; WO 03/87304; WO 04/26871; WO 04/26302; WO 04/24159, U.S. Pat. No. 6,184,226; WO 03/97639; and WO 04/16606); proteins involved in TGF-β signaling, including SMADs and MADs (EP 874046; WO 97/31020; WO 97/38729; WO 98/03663; WO 98/07735; WO 98/07849; WO 98/45467; WO 98/53068; WO 98/55512; WO 98/56913; WO 98/53830; WO 99/50296; U.S. Pat. No. 5,834,248; U.S. Pat. No. 5,807,708; and U.S. Pat. No. 5,948,639), Ski and Sno (Vogel, Science, 286:665 (1999); and Stroschein et al., Science, 286:771-774 (1999)); and any mutants, fragments, or derivatives of the above-identified molecules that retain the ability to directly inhibit the biological activity of TGF-β.

In some embodiments, the TGF-β antagonist is a direct TGF-β antagonist, for example an antibody that blocks TGF-β binding to its receptor. The antibody is such that it specifically binds to at least one isoform of TGF-β or to the extracellular domain of at least one TGF-β receptor. In some other embodiments, the anti-TGF-β antibody specifically binds at least one isoform of TGF-β selected from the group consisting of TGF-β1, TGF-β2, and TGF-β3. In yet other embodiments, the anti-TGF-β antibody specifically binds to at least: (a) TGF-β1, TGF-β2, and TGF-β3 (also referred to as “pan-neutralizing antibody”); (b) TGF-β1 and TGF-β2; (c) TGF-β1 and TGF-β3; and (d) TGF-β2 and TGF-β3. In various embodiments, the affinity constant Kₐ of the TGF-β antibody for at least one isoform of TGF-β, which it specifically binds, is preferably greater than 10⁶ M⁻¹, 10⁷ M⁻¹, 10⁸ M⁻¹, 10⁹ M⁻¹, 10¹⁰ M⁻¹, 10¹¹ M⁻¹, or 10¹² M⁻¹. In yet further embodiments, the antibody of the invention specifically binds to a protein substantially identical to human TGF-β1, TGF-β2, and/or TGF-β3. Also contemplated for use in humans are humanized forms and derivatives of nonhuman antibodies derived from any vertebrate species described in the cited references. Producing such variants is well within the ordinary skill of an artisan (see, e.g., Antibody Engineering, ed. Borrebaeck, 2nd ed., Oxford University Press, 1995).

In nonlimiting illustrative embodiments, the anti-TGF-β antibody is a murine monoclonal antibody 1D11 produced by the hybridoma 1D11.16 (ATCC Deposit Designation No. HB 9849, also described in U.S. Pat. Nos. 5,571,714; 5,772,998; and 5,783,185). The sequence of the 1D11 heavy chain variable region is available under accession No. AAB46787. Thus, in related embodiments, the anti-TGF-β antibody is a derivative of 1D11, e.g., an antibody comprising the CDR sequences identical to those in AAB46787, such as a humanized antibody. In yet further nonlimiting illustrative embodiments, the anti-TGF-β antibody is an antibody according to Lucas et al. J. Immunol. 145:1415-1422 (1990) or a fully human recombinant antibody generated by phage display, such as CAT192 described in WO 00/66631, U.S. Pat. No. 6,492,497, and U.S. Patent Application Publication Nos. 2003/0091566 and 2003/0064069, or an antibody comprising the CDR sequences disclosed therein. In yet further embodiments, the anti-TGF-β antibody is an antibody produced by guided selection from 1D11, CAT192, or CAT 152.

While the 1D11 antibody specifically binds all three mammalian isoforms of TGF-β, CAT192 specifically binds TGF-β1 only. The antigen affinities for 1D11 and CAT192 are approximately 1 nM and 8.4 pM, respectively. The epitopes for 1D11 (Dasch et al., J. Immunol. 142:1536-1541 (1998)) and CAT192 have been mapped to the C-terminal portion of mature TGF-β.

Methods for assessing neutralizing biological activity of TGF-β and TGF-β antagonists are known in the art. Examples of some of the more frequently used in vitro bioassays include the following:
(1) induction of colony formation of NRK cells in soft agar in the presence of EGF (Roberts et al., Proc. Natl. Acad. Sci. USA 78:5339-5343 (1981));
(2) induction of differentiation of primitive mesenchymal cells to express a cartilaginous phenotype (Seyedin et al., Proc. Natl. Acad. Sci. USA 82:2267-2271 (1985));
(3) inhibition of growth of Mv1Lu mink lung epithelial cells (Danielpour et al. (1989) J. Cell. Physiol., 138:79-86) and BBC-1 monkey kidney cells (Holley et al., Proc. Natl. Acad. Sci. USA 77:5989-5992 (1980));
(4) inhibition of mitogenesis of C3H/HeJ mouse thymocytes (Wrann et al., EMBO J. 6:1633-1636 (1987));
(5) inhibition of differentiation of rat L6 myoblast cells (Florini et al., J. Biol. Chem. 261:16509-16513 (1986));
(6) measurement of fibronectin production (Wrana et al., Cell 71:1003-1014 (1992));
(7) induction of plasminogen activator inhibitor I (PAI-1) promoter fused to a luciferase reporter gene (Abe et al., Anal. Biochem. 216:276-284 (1994));
(8) sandwich enzyme-linked immunosorbent assays (Danielpour et al., Growth Factors 2:61-71 (1989)); and
(9) cellular assays described in Singh et al., Bioorg. Med. Chem. Lett. 13(24):4355-4359 (2003).

### Uses and Methods of Administration

The methods of the invention comprise administering a TGF-β antagonist to a mammalian subject to treat, prevent, or reduce the risk of occurrence of a viral-associated lymphoproliferative disorder (LPD) and to treat proliferative disorders associated with low IFN-γ levels. In certain embodiments, methods for treating viral-associated disorders in individuals with low IFN-γ levels or individuals with an IFN-γ genotype associated with low IFN-γ levels are provided.

The invention further provides methods for assessing the presence of one or more risk factors for the presence or development of a viral-associated LPD, or its progression or responsiveness to treatment, and administering a TGF-β antagonist to a subject having the risk factor. For example, methods comprising assessing or measuring IFN-γ levels or IFN-γ genotype, and treating a subject with low IFN-γ levels or with the A/T or A/A+874 genotype are provided herein.

In certain embodiments, the viral-associated LPD is associated with infection by a herpes virus, e.g., HHV-8, cytomegalovirus, or Epstein-Barr virus (EBV). In other embodiments, the viral-associated disorder is associated with infection by a C-type retrovirus such as human T-lymphotropic virus type 1, for example. In other embodiments, the viral-associated disorder is associated with infection by a human immunodeficiency virus (e.g., HIV, HIV-1, HIV-2).

The disclosed methods include administering to a mammalian subject at risk for, susceptible to, or afflicted with a viral-associated LPD, therapeutically effective amounts of a TGF-β antagonist. The populations treated by the methods of the invention include, but are not limited to, subjects suffering from, or at risk for the development of, a viral-associated LPD or an LPD associated with low levels of IFN-γ, such as subjects with immune deficiency or viral infection.

Subjects treated according to the methods of the invention include but are not limited to humans, baboons, chimpanzees, and other primates, rodents (e.g., mice, rats), rabbits, cats, dogs, horses, cows, and pigs. Preferably, the subject will be a mammal. In other embodiments, the subject will be a human or a non-human mammal.

Many methods are available to assess development or progression of a viral-associated LPD, and to evaluate inhibitors thereof. An LPD is a disease or condition that involves aberrant proliferation of lymphocytes or lymphatic tissues, i.e. a “viral-associated lymphoproliferative disorder,” “EBV-associated LPD,” or “post-transplant lymphoproliferative disorder,” for example. Such disorders include, but are not limited to, any acute or chronic disease or disorder as defined above.

Development or progression of an LPD may be assessed by adenopathy (swollen or enlarged lymph nodes), spenomegaly, or symptoms attributable to organ infiltration by an expanding lymphoid clone, such as abdominal bloating (gastrointestinal tract), or pulmonary abnormalities (lungs). Symptoms of PTLD include fever, night-sweats, and weight loss, for example. The presence or progression of an LPD may also be detected by computed topomography (CT) scans of the chest, abdomen, and pelvis; gallium-67 single photon emission computed tomography (SPECT) scan, bone marrow aspirate and biopsy; and evaluation of liver and kidney function, blood serum tumor markers, and serum lactate dehydrogenase (LDH), for example.

The presence of EBV or other virus (latent or active infection) may be detected by techniques known in the art, including but not limited to in situ hybridization for viral RNA or immunohistochemistry, such as for latent membrane protein-1 of EBV. Further, in situ reverse transcription-polymerase chain reaction (IS-RT-PCR) may be used to detect latent or active viral infection, for example using forward and reverse primers for a viral protein, such as EBV thymidine kinase primers (Porcu et al., Blood 100:2341-2348 (2002)).

An LPD is characterized by aberrant lymphocyte proliferation. Methods to detect aberrant proliferation, function, or structure of a lymphatic (or other) cell or tissue may be used to diagnose, monitor the progression of, or assay the efficacy of a therapeutic agent for an LPD. Lymphocyte proliferation may be measured with flow cytometry or other means to determine total T or B cell numbers, CD8+ cells, and cell-based assays of T cell proliferation. Lymphocyte state and proliferation may also be measured by cell-based assays of responsiveness to antigen challenge, such as a mixed lymphocyte reactivation assay, or by measuring the presence of activation antigens such as CD25, CD69 and/or CD71 on T cells, for example.

A method of the invention may reduce aberrant lymphocyte proliferation or accumulation by at least 10%, 20%, 30%, 40%, 50%, 60%, 70%, 80%, 90%, 95% or more. In some embodiments, the invention provides a method of treating or ameliorating a viral-associated lymphoproliferative disorder, to allow one or more symptoms of the subject's lymphoproliferative disorder to improve by at least 10%, 20%, 30%, 40%, 50%, 60%, 70%, 80%, 90%, 100% or more. Other indications for treatment include, but are not limited to, the presence of one or more risk factors for an LPD, or PTLD, including those discussed previously, and in the following sections. A subject at risk for developing or susceptible to a disorder or a subject who may be particularly receptive to treatment with a TGF-β antagonist may be identified by ascertaining the presence or absence of such one or more risk factors.

### Cytokine Genotype

A subject is at risk for developing or susceptible to a viral-associated lymphoproliferative disorder, an LPD, or a PTLD, if they are homozygous or heterozygous for a low producer IFN-γ genotype, such as an A/A or A/T genotype at position +874 of the IFN-γ gene. Methods to assess the relative cytokine production level of various cytokine polymorphisms include ex vivo cytokine production assays using stimulated peripheral blood mononuclear cells (PBMCs). Accordingly, in studies of ex vivo IFN-γ production of the IFN-γ polymorphism at +874, the low producer A/A genotype shows an approximately 40%, 50%, 60%, 70%, or 80% reduction in IFN-γ level. IFN-γ levels may be measured in the supernatants of cells cultured in PPD-stimulated cells minus IFN-γ in supernatants of cells cultured in media alone as compared to the T/T genotype cells.

### Cytokine Levels

The methods disclosed may be useful in subjects with circulating IFN-γ levels of less than 100, 90, 80, 70, 60, 50, 40, 30, 20, 10, 8, 6, 5, or 4 pg/mL. Furthermore, the treatment may be useful in subjects with circulating TGF-β levels of at least 5, 6, 7, 8, 9, 10, 12, 14, 16, 18, 20, 25, 30, 40, 50, 60, 70, 80, 90, or 100 ng/mL or more, when the increase in TGF-β levels is associated with or caused by a lymphoproliferative disorder. TGF-γ or IFN-γ levels (total or active) may be measured in body fluids such as blood, serum, or urine, for example. In some embodiments, the claimed methods include administration of a TGF-β antagonist to allow reduction of circulating TGF-β levels in a subject to undetectable levels, or to less than 1%, 2%, 5%, 10%, 20%, 30%, 40%, 50%, 60% or 70% of the subject's TGF-β level prior to treatment. Similarly, the claimed methods include administration of a TGF-β antagonist to allow increases in circulating IFN-γ levels of at least 20%, 30%, 40%, 50%, 60%, 70%, 80%, 90%, 100%, 150%, 200%, 300% or more. Cytokine serum levels are measured, for example, with enzyme immunoassay techniques, such as sandwich ELISA assays, and as described herein.

One skilled in the art would appreciate that gene polymorphisms within the IFN-γ gene or other genes, the products of which affect IFN-γ levels, are one of several mechanisms by which IFN-γ production, or other cytokine levels, could be influenced. Other factors influencing IFN-γ level include other polymorphisms within the IFN-γ gene, or transcriptional, post-transcriptional, or post-translational mechanisms that influence IFN-γ production.

Normal human IFN-γ serum levels are at or about 30 pg/ml+/−10 pg/ml, but IFN-γ levels vary with lymphocyte levels and IFN-γ genotype, for example. IFN-γ levels increase under pathologic circumstances such as trauma, infection, cancer, and autoimmunity. TGF-β concentrations in normal human fluids are at or about 5 ng/mL TGF-β1 in plasma and 300 pg TGF-β1/mg creatinine in urine. In normal human plasma TGF-β2 and TGF-β3 levels are less than 0.2 ng/mL.

### Immune Deficiency and Transplantation

A subject with an immune deficiency or a subject who had or is having an organ, tissue, or cell transplant is at risk for an LPD, for example. The incidence of PTLD varies with the organ or tissue transplanted, and examples of transplant include heart, kidney, lung, liver, cornea, bone marrow, stem cell, blood vessel, and islet cell transplant. Immunosuppressive therapy associated with transplantation will place a subject at risk for an LPD. Further risk factors for development of an LPD such as PTLD in a transplantation subject, include the absolute and relative T cell number, the CD8+ T cell number, a change in T cells, such as CD8+ cells over time, the type of transplanted organ, EBV sero-negative status, EBV viral load, age of the subject (i.e., child or adult), the type and duration of immunosuppressive therapy administered to prevent graft rejection, the degree of immunosupression, and the degree of major histocompatability (MHC) mismatch, among others. Transplant recipients under 5 years of age, under 10 years or age, under 15 years of age, or under 18 years of age are at increased risk of developing an LPD such as a PTLD. Bone marrow or lung transplant recipients have a 20% incidence of PTLD, and renal transplant recipients have a PTLD incidence of 1-2%. Primary EBV infection occurring at or after an organ, tissue, or cell transplant places a subject at risk for an LPD. Particularly, if the transplant donor is EBV+, but the recipient is EBV−, primary viral infection is associated with an increased risk of PTLD. EBV or other viral infection in an immune deficient subject places the subject at risk for an LPD. A subject at risk may be identified, for example, by evaluating viral loads in blood and tissues (for example looking for increased viral load after transplant), or by testing for increased numbers of leukocytes, B cells, or total serum IgM. EBV (or other virus) may be detected by Southern blot hybridization or by polymerase chain reaction (PCR), including quantitative or semiquantitative PCR, or by positive viral serology (anti-viral capsid antigen IgG (EBV serology)) in the blood, serum, or tissue of a subject, as appropriate. EBV strain infecting the different donors and the donors' atopic status are other possible risk factors for LPD development.

The methods of the invention may be useful in subjects with immune deficiency. For example, the methods of the invention can be used to treat or prevent one or more LPDs in subjects with an immune deficiency where immune function is below normal by 25%, 40%, 50%, 60% 75%, 80%, 90% or more. The methods may be used in subjects having T cell counts, CD8+ cell counts, CD3+/CD8+ cell counts, or EBV-specific T cell counts of less than 500, 400, 300, 200, 100, 75, 50, 25, or 10 cells/μL, for example.

### Immunosuppressive Agents

Immune deficiency may result from administration of an immunosuppressive agent. The terms “immunosuppressive agent,” “immunosuppressant,” and “immunodepressant” as used herein, refer to a compound or composition that induces immunosuppression, i.e., it prevents or interferes with the development of immunologic response. Example of immunosuppressive agents include, but are not limited to, Sandimmune™, Neoral™ (cyclosporine); Prograf™, Protopic™ (tacrolimus); Rapamune™ (sirolimus); SZD-RAD, FTY720; Certican™ (everolimus, rapamycin derivative); campath-1H (anti-CD52 antibody); Rituxan™ (rituximab, anti-CD20 antibody); OKT4; LEA29Y (BMS-224818, CTLA4Ig); indolyl-ASC (32-indole ether derivatives of tacrolimus and ascomycin); Imuran™ (azathioprine); Atgam™ (antithymocyte/globuline); Orthoclone™ (OKT3; muromonab-CD3); Cellcept™ (mycophenolate mofetil); Thymoglobulin®; Zenapax™ (daclizumab); Cytoxan™ (cyclophosphamide); prednisone, prednisolone and other corticosteoids malononitrilamides (MNAs (leflunomide, FK778, FK779)); and 15-deoxyspergualin (DSG).

Methods for assessing immunosuppressive activity of an agent are known in the art. The length of the survival time of the transplanted organ in vivo with and without pharmacological intervention serves as a quantitative measure for the suppression of the immune response. In vitro assays may also be used, for example, a mixed lymphocyte reaction (MLR) assay (see, e.g., Fathman et al., J Immunol., 118:1232-1238 (1977)); a CD3 assay (specific activation of immune cells via an anti-CD3 antibody (e.g., OKT3)) (see, e.g., Khanna et al., Transplantation, 67:882-889 (1999); Khanna et al., Transplantation, 67:S58 (1999)); and an IL-2R assay (specific activation of immune cells with the exogenously added cytokine IL-2) (see, e.g., Farrar et al., J. Immunol., 126:1120-1125 (1981)).

### Therapeutic Methods

Administration of TGF-β antagonists in accordance with the methods of the invention is not limited to any particular delivery system and may include, without limitation, parenteral (including subcutaneous, intravenous, intramedullary, intraarticular, intramuscular, or intraperitoneal injection) rectal, topical, transdermal, or oral (for example, in capsules, suspensions, or tablets). Administration to an individual may occur in a single dose or in repeat administrations, and in any of a variety of physiologically acceptable salt forms, and/or with an acceptable pharmaceutical carrier and/or additive as part of a pharmaceutical composition (described earlier). Physiologically acceptable salt forms and standard pharmaceutical formulation techniques and excipients are well known to persons skilled in the art (see, e.g., Physician's Desk Reference (PDR) 2003, 57th ed., Medical Economics Company, 2002; and Remington: The Science and Practice of Pharmacy, eds. Gennado et al., 20th ed, Lippincott, Williams & Wilkins, 2000).

Administration of an antagonist to an individual may also be accomplished by means of gene therapy, wherein a nucleic acid sequence encoding the antagonist is administered to the patient in vivo or to cells in vitro, which are then introduced into a patient, and the antagonist (e.g., antisense RNA, soluble TGF-β receptor) is produced by expression of the product encoded by the nucleic acid sequence. Methods for gene therapy to deliver TGF-β antagonists are known to those of skill in the art (see, e.g., Fakhrai et al., Proc. Nat. Acad. Sci. U.S.A., 93:2909-2914 (1996)).

In the disclosed methods, a TGF-β antagonist may be administered alone, concurrently or consecutively over overlapping or nonoverlapping intervals with one or more additional biologically active agents, such as an anti-viral agent. Examples of antiviral agents include but are not limited to acyclovir, ganciclovir, and foscarnet, and the like. Additional biologically active agents may include immunosuppressive agents, anti-B-cell monoclonal antibodies, and EBV-specific autologous CTLs, and the like. A TGF-β antagonist may be administered concurrently with a reduction in immunosuppressive therapy, for example, to treat a subject with PTLD. In sequential administration, the TGF-β antagonist and the additional agent or agents can be administered in any order. In some embodiments, the length of an overlapping interval is more than 2, 4, 6, 12, 24, or 48 weeks.

The antagonists may be administered as the sole active compound or in combination with another compound or composition. Unless otherwise indicated, the antagonist is administered as a dose of approximately from 10 μg/kg to 25 mg/kg, depending on the severity of the symptoms and the progression of the disease. Most commonly, antibodies are administered in an outpatient setting by weekly, bimonthly, or monthly administration at about 0.1-15 mg/kg doses by slow intravenous (IV) infusion. The appropriate therapeutically effective dose of an antagonist is selected by a treating clinician and would range approximately from 10 μg/kg to 20 mg/kg, from 10 μg/kg to 10 mg/kg, from 10 μg/kg to 1 mg/kg, from 10 μg/kg to 100 μg/kg, from 100 μg/kg to 1 mg/kg, from 100 μg/kg to 10 mg/kg, from 500 μg/kg to 5 mg/kg, from 500 μg/kg to 20 mg/kg, from 1 mg/kg to 5 mg/kg, from 1 mg/kg to 25 mg/kg, from 5 mg/kg to 50 mg/kg, from 5 mg/kg to 25 mg/kg, and from 10 mg/kg to 25 mg/kg. Additionally, specific dosages indicated in the Examples or in the Physician's Desk Reference (PDR) 2003, 57th ed., Medical Economics Company, 2002, may be used.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

Association of IFN-γ genotype with PTLD—Clinical observations: The cytokine genotypes of 12 PTLD patients were analyzed, further to a preliminary evaluation of cytokine genotype in 9 PTLD patients that has been reported previously (VanBuskirk et al., Transplant. Proc. 33:1834 (2001)). The cytokine genotyping of the 12 PTLD patients shows that the proportion of patients with the A/A genotype for the IFN-γ gene is higher in PTLD patients than in 135 non-PTLD transplant patients at the same transplant center (58% versus 27%, p=0.02). In this study, observation of genotype distributions for TGF-β, IL-6, IL-10 and TNF-α, shows no statistically significant differences between PTLD and non-PTLD patients. This work identifies the IFN-γ A/A genotype as a risk factor in PTLD.

Analysis of subject genotype and other factors associated with LPD: To assess a subject or donor's genotype, genomic DNA was isolated from PBL using Qiagen (Valencia, Calif.) DNA extraction kits. HLA analysis was done using Pel-Freez Clinical Systems AB/DR PCR-SSP unitrays (Brown Deer, Wis.). Cytokine genotyping for TGF-β, TNF-α, IL-6, IL-10, and IFN-γ was accomplished using Cytgen cytokine genotyping trays from One Lambda (Canoga Park, Calif.). PCR products were run on 2% agarose gels and visualized with ethidium bromide. Banding patterns were interpreted using manufacture's templates and compared to internal controls in each lane.

Subjects and PBL donors were tested for EBV reactivity by ELISA (Meridian, Cincinnati, Ohio) and EBV-reactive trans vivo DTH assays prior to injection into SCID mice or use in CTL restimulation cultures.

To evaluate T cells and T cell subsets, flow cytometry is used on fresh blood samples by standard 3-color flow cytometry. EBV-reactive CD8+ T cells are detected by flow cytometry using HLA-B8 tetramers complexed with immunodominant EBV peptides derived from the latent gene, EBNA-3A, or the immediate early lytic gene BZLF-1. Frozen patient peripheral blood mononuclear cells (PBMCs) are viably thawed, incubated overnight at 37° C., and then purified by Ficoll-Hypaque density gradient centrifugation to remove debris. Cells are stained with phycoerythrin (PE)-conjugated murine anti-CD8 and fluorescein isothiocyanate (FITC)-conjugated murine anti-CD3 antibodies (both from BD Pharmingen, San Diego, Calif.) and allophycocyanin (APC)-conjugated HLA-matched tetramer reagent or a nonreactive control. Approximately 10⁵ lymphocyte-gated (based on forward and side scatter) events are collected for each flow analysis.

### Example 2

Association of IFN-γ genotype with LPD development in hu-PBL SCID mice: The hu PBL-SCID mouse, in which human (hu) peripheral blood leukocytes (PBL) from healthy EBV sero-positive donors are injected into SCID mice, is a reproducible model of spontaneous EBV-driven lymphoproliferative disease (LPD). EBV-positive B cell tumors arising in hu PBL-SCID mice are phenotypically and genotypically very similar to PTLD (Picchio et al., Cancer Research 52:2468-2477 (1992); Baiocchi et al., Proc. Natl. Acad. Sci. U.S.A. 91:5577-5581 (1994)). In this model system, LPD production and development varies between donors—a heterogeneity that has not been extensively studied (see Picchio et al., supra; Mosier et al., AIDS Res. Hum. Retroviruses 8:735-740 (1992); Coppola et al., J. Immunol. 160:2514-2522 (1998)).

Murine NK cells are also known to influence LPD development (Baiocchi et al., supra; Lacerda et al., Transplantation 61:492-497 (1996)), as are murine macrophages (Yoshino et al., Bone Marrow Transplant. 26:1211-1216 (2000)), and it is possible that differential ability to activate murine NK cells could account for some heterogeneity in LPD development. NK cells were purposefully not depleted or neutralized in this study, to make the model more stringent. Thus, any observed association of cytokine polymorphism and LPD indicate a strong association.

The hu PBL-SCID mouse model of LPD is as follows: Female Balb/c or CB. 17 scid/scid (SCID) mice were purchased from Charles River or Taconic. Mice were housed and treated in accordance with NIH and institutionally approved guidelines. Mice received 50×10⁶ human PBL intraperitoneally in saline. PBL were obtained from American Red Cross leukopacks, or from volunteers using institutional review board approved protocols. PBL were isolated by ficoll-hypaque according to standard methods. PBL from each donor were injected into three to five separate mice. Human PBL engraftment was monitored with bi-weekly ELISAs for the presence of human IgG in mouse serum, as previously described (Baiocchi et al., Proc. Natl. Acad. Sci., U.S.A. 91:5577-5581 (1994)). Mice included in this study had >750 μg/ml of human IgG, which increased to >1 mg/ml when tumors were detected. Latency was defined as the time after injection until mice became moribund or died (Picchio et al., Cancer Research. 52:2468-2477 (1992)). All animals were inspected at death for the presence of tumors, and these tumors confirmed to be of human B cell origin using flow cytometry. Only mice with confirmed human tumors were considered to have LPD.

In the hu PBL-SCID mouse study, PBL from each of 49 EBV-reactive donors were injected into 3 to 5 SCID mice per donor. Recipient mice were monitored for up to 6 months for engraftment by human cells (as evidenced by human IgG in the serum) and development of LPD (human CD45+CD19/CD20+ tumors infiltrated with small numbers of CD45+CD3+ cells). As shown in Table 1, PBL from 47% (23 of 49) of the donors produced no LPD after 20 weeks, while 24% (12 of 49) developed LPD tumor rapidly (median time to LPD, 8 weeks) and with high penetrance (median 100%, range 80-100%). PBL from the remainder of the donors (29%, 14 of 49) produced LPD later (median 12 weeks), and in fewer mice (median penetrance 55%, range 33-100%). As determined by the exact Wilcoxon Rank Sum test, the differences in latency and penetrance between the rapid and intermediate/late groups are statistically significant (p<0.0001).

TABLE 1
LPD development and penetrance.

| | Median onset | Range | Median LPD | Range |
|---|---|---|---|---|
| Donor Group | (weeks) | (weeks to LPD) | Penetrance | (LPD Penetrance) |
| Rapid LPD (n = 12) | 8* | 6-10 | 100** | 80-100% |
| Late LPD (n = 14) | 12 | 10-18 | 55 | 33-100% |
| No LPD (n = 23) | >20 | Not applicable | Not applicable | Not applicable |

*p < .0001 compared to late LPD group
**p < .0001 compared to late LPD group

In the hu PBL-SCID mouse model of spontaneous EBV-LPD, cytokine genotype data on 49 donors demonstrates that donor-derived variability in LPD development correlates with IFN-γ genotype. Fifty-three percent of the EBV-seropositive donors in this study produced LPD in the hu PBL-SCID mice within 6 months. Of donors producing LPD, 12 rapidly produced LPD (median time to LPD, 8 weeks) with high penetrance (median 100%). The other LPD producer phenotype developed LPD later (median time 12 weeks) and with lower penetrance (median 55%).

To determine if cytokine genotypes correlate with LPD development, the distribution of cytokine genotypes for IFN-γ, TNF-α, IL-6, IL-10 and TGF-β in the PBL used to produce EBV-LPD in hu PBL-SCID mice was studied. Rapid, high penetrance LPD producers were compared with intermediate/late LPD producers and with donors whose PBL did not produce LPD (as determined in Table 1). Table 2 demonstrates that analysis of the distribution of polymorphisms for IFN-γ demonstrated statistically significant differences between rapid LPD producers and the other two groups. Of the 12 rapid LPD producers, none were of the T/T genotype, 5 were T/A genotype (41.7%), and 7 were A/A genotype (58.3%). In contrast, donors whose PBL produced intermediate/late LPD or not at all, exhibited a more heterogeneous distribution of genotypes (14 T/T, 37.8%; 15 T/A, 43.3% and 8 A/A, 18.9%).

TABLE 2
IFNγ genotypes and LPD development in hu PBL-SCID mice^{A}

| | | Intermediate/ | |
|---|---|---|---|
| | Rapid LPD | Late LPD | No LPD |
| Genotype | N = 12 | N = 14 | N = 23 |
| A/A* | 58.3% | 7.1% | 26.1% |
| T/A | 41.7% | 50.0% | 39.1% |
| T/T** | 12 0% | 42.9% | 34.8% |

3-5 SCID mice were injected per PBL donor, and all mice were engrafted, as evidenced by >750 μg/ml human IgG in the sera.
*A/A genotype is significantly more prevalent in the Rapid LPD group, p = 0.0144.
**the presence of the A allele (A/A + T/A) is significantly more prevalent in the Rapid LPD group, p = .0257

Statistical analyses of these data indicate that the A/A genotype was significantly more frequent in the rapid LPD producers compared to the intermediate/late LPD producers and the no LPD producers (p=0.0144). The absence of the T/T genotype among the rapid LPD producers suggests that the presence of the T allele correlated with a lack of LPD development in hu PBL-SCID mice. All (12 of 12) of the rapid LPD producers had at least one A allele present, contrasted to the intermediate/late LPD producers (8 of 14) and no LPD producers, where 15 of 23 donors had at least one A allele present. This is a statistically significant difference between the three groups (p=0.0257). When the cytokine polymorphism distributions for TNF-α, IL-6, TGF-β and IL-10 were analyzed, no statistical differences were observed between the groups of donors. Similar to the reported distributions for TGF-β genotypes (Perrey et al., Transplant Immunology 6:193-197 (1998)), the majority of the donors exhibited genotypes for high TGF-β production. Indeed, 48 of the 49 PBL donors, and all of those producing rapid LPD had genotypes linked to high TGF-β production.

Importantly, these data show that the A (adenosine) allele for IFN-γ at base +874 is strongly associated with LPD production. Of the rapid, high penetrance LPD donors, 58% were homozygous for the A allele (A/A), while 42% were heterozygous (T/A). None of the rapid, high frequency LPD producers were homozygous for the T allele. In contrast, all genotypes were represented in the groups of donors who produced LPD late or not at all. The frequency of the A/A genotype among the rapid LPD producers was significantly different compared to the intermediate/late LPD producers, and the no LPD donors (p=0.0144). Also significant (p=0.0257) is the presence of the A allele in rapid LPD producers compared to the other 2 LPD groups. These data mirror the clinical observations of PTLD patients, suggesting that the IFN-γ genotype association with LPD production in hu PBL-SCID mice is a risk factor or indicator of clinical significance.

### Example 3

Cytokine Production of IFN-γ and TGF-β Genotypes: The A/A, T/A and T/T IFN-γ genotypes for base +874 have been reported to correspond to low, intermediate and high cytokine in vitro production respectively (Pravica et al., Hum. Immunol. 61:863-866 (2000); Hoffmann et al., Transplantation 72:1444-1450 (2001); Lopez-Maderuelo et al., Am. J. Respir. Crit. Care Med. 167:970-975 (2003)). We observed a clear-cut association of genotype with cytokine production when the same antigenic stimulus was provided, i.e., in tests of HLA-A, -B matched donors using the same EBV-LCL. Of the four donors that met these criteria, the A/A genotype donor produced the least IFN-γ (4,928+/−1,795 pg/ml), with the 2 A/T genotype donors producing an intermediate amount of cytokine (25,945+/−958 pg/ml) and the 1 T/T genotype donor producing the most IFN-γ (41,312+/−1,811 pg/ml). Administering TGF-β at 10 ng/ml to the supernatent of these cultures reduced IFN-γ production by approximately 68%, 35%, and 66%, respectively.

In a prior published study of IFN-γ production by the +874 polymorphism genotypes, ex vivo cytokine production was assayed, obtaining PBMCs from venous whole blood (20 ml) from individuals (López-Maderuelo et al., supra). Cells were cultured at a concentration of 2.0×10⁶ cells/ml and were stimulated with a purified protein derivative (PPD) antigen (10 pg/ml; Statens Seruminstitut, Copenhagen, Denmark) for 96 hours at 37° C. with 5% CO₂. Culture supernatants were harvested and assayed with ELISA kits for IFN-γ (Biosource International, Camarillo, Calif.). The assays presented a detection limit of 4 pg/ml; interassay and intra-assay coefficients of variation were less than 10%. The A/A +874 genotype produced IFN-γ levels of approximately 600 pg/mL, while the TA/TT genotypes produced IFN-γ levels of approximately 1200 pg/mL, with the IFN-γ levels presented as the concentration in supernatants of PPD-stimulated cells minus the concentration in supernatants of cells cultured in media alone (López-Maderuelo et al., supra).

Low levels of IFN-γ production are therefore associated with the A (adenosine) at +874 polymorphism, and may serve as an independent risk factor associated with proliferative disorders, such as viral-associated LPD or PTLD. Additional causes of low IFN-γ production, are contemplated, and encompassed by the claimed methods.

Similarly, genotypes having high TGF-β production may be identified and assessed. As noted above, the majority of PBL donors in this study exhibited TGF-β genotypes associated with high production and all of those producing rapid LPD had genotypes linked to high TGF-β production (see Perrey et al., Transplant Immunology 6:193-197 (1998)).

### Example 4

TGF-β inhibition of CTL activity is associated with IFN-γ genotype: To further examine the relationship between IFN-γ genotype and CTL function, we next tested whether TGF-β could inhibit re-stimulation of CTL activity in vitro. PBL were cultured with irradiated HLA-matched LCL stimulators in the presence or absence of TGF-β1 for 5 days. CTL activity was assessed using standard CTL assays.

Detecting CTL activity against EBV antigens requires a 5-12 day restimulation culture (Vooijs et al., Scand. J. Immunol. 42:591-597 (1995)). PBL were cultured with HLA-A, -B matched LCL in the absence or presence of 10 ng/ml TGF-β for 5 days. Viable cells were washed three times to remove any exogenous TGF-β and CTL activity was assessed using standard lysis assays, and as described herein.

Cytolysis Assays: Standard non-radioactive cytotoxicity assays were set up using PBL from 5 to 7-day re-stimulation cultures and either HLA-matched or mismatched LCL lines at various effector-to-target ratios, with target cells plated at 5×10⁴ to 1×10⁵ cells/ml. All samples were plated in triplicate. Alamar blue (Biosource, Carmillo, Calif.) was used at a dilution of 1:10. Cells were cultured for 24 hours, and read on a Cytofluor II fluorescent multi-well plate reader (Perspective Biosystems) at an excitation wavelength of 530 nm and an emission wavelength of 590 nm. Percent lysis was determined as follows: {targets alone−[(E+T)−(E alone)]/targets alone}. Lytic units (LU) are arbitrarily defined as the number of lymphocytes required to yield the selected lysis value (in this case, 30%). To define LU, all curves must pass through this lysis value, and it must be in the linear portion of the curve. The number of LU per million cells is calculated using the following formula: LU per million cells=106/[(# effectors/percent lysis)×(30)].

Data are shown as percent control lysis of PBL cultured with LCL in the absence of TGF-β. For each donor, multiple effector to target ratios were tested in triplicate, and LU determined from the linear portions of the curves. The percent inhibition was calculated using LU from control versus TGF-β treated cultures. The results shown are the mean and standard deviation for the triplicates from representative experiments for each donor. When analyzed by t-test, the CTL activity in A/A and T/A PBL restimulated in the presence of TGF-β is significantly different from either control CTL activity or the CTL activity in T/T PBL after culture with TGF-β (p=0.015). The T/T genotype can, in some instances, confer a “PTLD” phenotype in the mouse-human chimeric model, leading to rapid development of LPD in this model. Further, TGF-β antagonists are effective to increase survival in the hu PBL SCID mouse model using T/T donor PBL that produce rapid and/or high penetrance LPD.

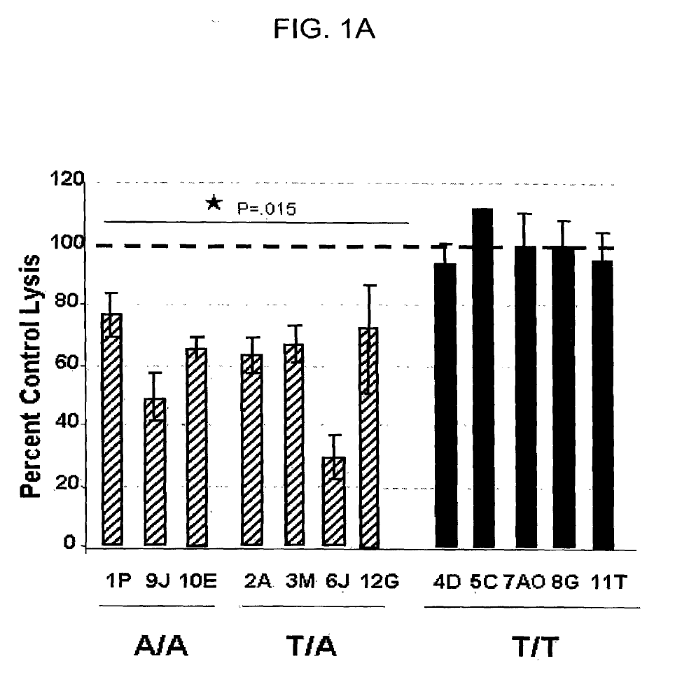

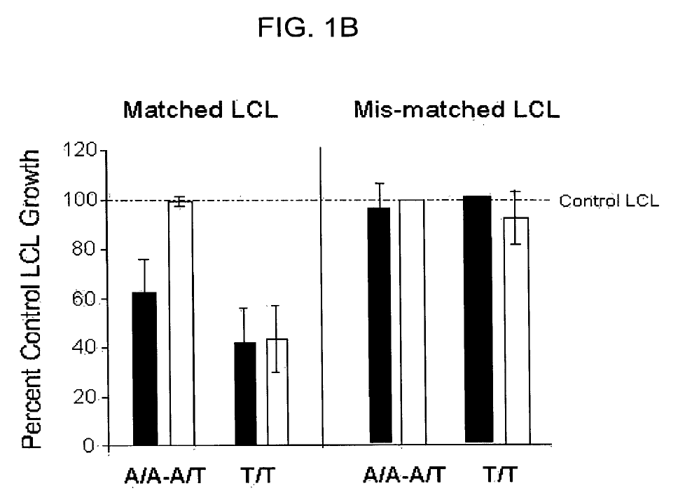

FIG. 1A shows that PBL from individuals with the A/A or A/T IFN-γ genotype had an impaired CTL response if TGF-β was added to the re-stimulation cultures. TGF-β-treated cultures for these donors had 25-70% inhibition of cytolysis compared to control cultures. In contrast, TGF-β had no detected effect on CTL restimulation of T/T genotype PBL in this assay. Data are shown as the mean percent of control lysis, determined using lytic units (LU). The difference between the A/A+T/A genotype cultures and the T/T genotype cultures was significant (p=0.015).

In this study CTL were restimulated efficiently in vitro regardless of the IFN-γ genotype (not shown), indicating that a lack of CTL precursors or a generalized defect in CTL restimulation could not explain the association of the A/A genotype with LPD development. These data show that when TGF-β was present, CTL restimulation was significantly reduced in A/A or A/T genotype PBL, genotypes associated with PBL that produce rapid and/or high penetrance LPD in this model.

### Example 5

Activity of TGF-β antagonists in growth inhibition assays: The effect of TGF-β on the inhibition of CTL re-stimulation using two-week LCL growth inhibition assays, similar to those described by Wilson et al. (Wilson et al., Clin. Exp. Immunol. 126:101-110 (2001)) was assayed next. Growth inhibition assays assess the ability of a set number of re-stimulated CTL to lyse a titrated number of LCL under more stringent conditions than regular CTL assays. LCL not killed by the CTL will proliferate and detectable differences in metabolic activity are seen after two weeks.

FIG. 1B shows that the ability of CTL to prevent matched LCL growth is inhibited by CTL re-stimulation in the presence of TGF-β. CTL were re-stimulated in the presence or absence of 10 ng/ml TGF-β. At the end of 5 days, CTL activity was assessed by standard CTL assays as in FIG. 1A. In addition, a portion of the re-stimulated cells (10⁴/well) were cultured with titrated numbers of HLA-A, -B matched or mis-matched LCL for 2 weeks. Data are shown as the mean percent LCL growth ±SD in wells containing both CTL and LCL compared to growth in wells containing only LCL as determined by alamar blue. Data are combined for 3 donors of each genotype at an 8:1 effector to target ratio. Solid bars: control CTL re-stimulated in the absence of TGF-β. Open bars: CTL re-stimulated in the presence of TGF-β.

These data indicate that CTL inhibited long term growth of matched but not mismatched LCL, and that A/A or A/T genotype CTL (n=3 donors) re-stimulated in the presence of TGF-β did not inhibit growth of their matched LCL targets. In this assay, the T/T genotype CTL re-stimulated in the presence of TGF-β (n=3 donors) inhibited LCL growth similarly to control CTL. As the T/T genotype is less commonly associated with disease state, a T/T donor demonstrating rapid and/or high penetrance LPD in this model was recently identified. Preliminary studies indicate that T/T cells producing rapid LPD are sensitive to TGF-β in this assay. Thus, the assays described above detected TGF-β inhibition of CTL restimulation.

### Example 6

Treatment with TGF-β antagonist prolongs survival of hu PBL SCID mice: In vivo treatment with anti-TGF-β improves survival of hu PBL SCID mice. Like the majority of the general population, all of the rapid LPD donors exhibited genotypes linked to high TGF-β production. Based on the in vitro data indicating that TGF-β could inhibit CTL restimulation, the effect of treatment with anti-TGF-β on survival of hu PBL SCID mice was investigated. These data show that reducing TGF-β in hu PBL SCID mice prolongs survival.

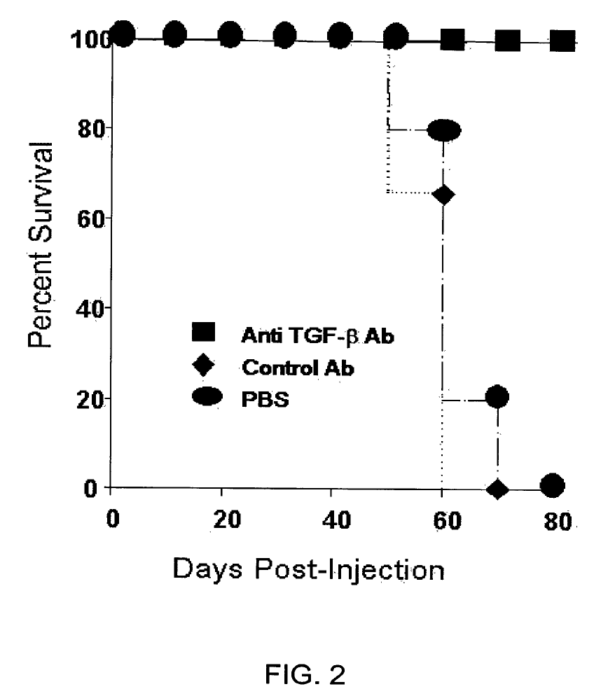

As demonstrated in FIG. 2, a survival trial using anti-TGF-β antibody resulted in 100% survival greater than 80 days in the anti-TGF-β treated mice. In contrast, all control animals died within 70 days. These data indicate an important role of TGF-β in LPD development.

In this study, SCID mice were injected with 50 million PBL as described in Example 2. Animals received either PBS (n=3), isotype 100 μg control antibody (n=5) or 100 pg anti-TGF-β (n=5) every other day for the duration of the experiment. Animals were confirmed to be engrafted by the presence of >750 μg/ml human IgG in their sera, and were monitored for LPD development. Survival time was determined for each group. When animals died or became moribund, flow cytometry was performed to confirm the development of LPD. As shown, all control animals (PBS or isotype control antibody) died within 70 days, while animals treated with anti-TGF-β antibody survived greater than 80 days. The differences in survival were highly significant (p=0.004 for PBS vs. anti-TGF-β and p=0.002 for Isotype control vs. anti-TGF-β).

Hu PBL-SCID mice were injected intraperitoneally with 100 μg of PBS, isotype control antibody or a commercially available anti-TGF-β antibody (Genzyme) three times per week for the duration of the experiment. All animals were engrafted, as evidenced by >750 μg/ml human IgG in the sera at 4 weeks post injection (not shown). As shown in FIG. 2, animals treated with either PBS or isotype control antibody had a mean survival of 60 days. In contrast, animals treated with anti-TGF-β survived greater than 80 days. Thus, anti-TGF-β treatment significantly enhanced survival of hu PBL-SCID mice (p<0.002).

### Example 7

In vivo Neutralization of TGF-β reduces LPD and results in CD8+ expansion and activation: To investigate the mechanism by which in vivo treatment with anti-TGF-β antibody prolonged survival, and to assess the utility of anti-TGF-β treatment, a second experiment using the A411 anti-TGF-β antibody and a second PBL donor was performed. In this protocol TGF-β levels, LPD development, and CD8 T cell expansion were evaluated. Hu PBL-SCID mice were initially injected with anti-TGF-β antibody three times per week and human Ig levels, serum TGF-β, and LDP development were monitored. All animals were engrafted, as evidenced by >750 μg/ml human IgG in the sera at 4 weeks post injection (not shown). Hu PBL-SCID mice routinely exhibited circulating levels of 12000 pg/ml TGF-β. Treatment of the animals with anti-TGF-β significantly reduced that level to less than 4000 pg/ml (p<0.05).

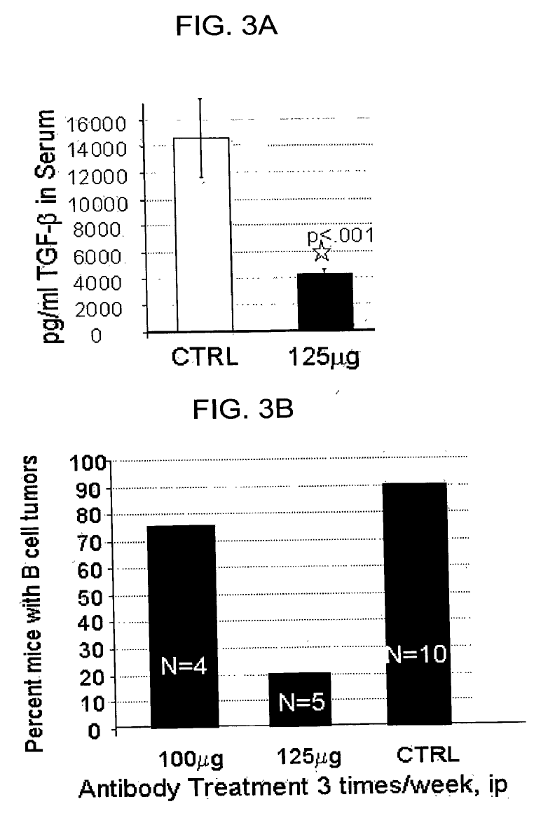

FIG. 3A shows that anti-TGF-β neutralizes TGF-β in vivo. To assess an effect on TGF-β levels, hu PBL-SCID mice were injected with 125 μg anti-TGF-β antibody (A411) or PBS three times per week. Serum samples were tested at week 6 for the presence of TGF-β by ELISA. The data of FIG. 3A are shown as mean pg/ml of TGF-β derived from triplicate determinations, 5 mice per group.

LDP development was next determined. Animals were sacrificed at 9 weeks, at which point 100% of the control animals had developed human B cell tumors. In contrast, only 20% (1 of 5) of the animals receiving 125 μg anti-TGF-β developed LPD (FIG. 3B). FIG. 3B shows that anti-TGF-β reduces the incidence of LPD in a dose dependent manner. Hu PBL-SCID mice were treated with 100 μg or 125 μg anti-TGF-β antibody A411 or mouse IgG three times per week for 9 weeks. At harvest, the presence of B cell tumors was assessed visually and confirmed by flow cytometry.

Splenocytes and tumor cells from hu PBL SCID mice were analyzed via flow cytometry to assess CD8+ T cell levels and T cell activation as described in Example 1. All antibodies and isotype control antibodies were directly conjugated and obtained from BD Pharmingen (San Diego, Calif.). Samples were read on a FACScan (BD) and analyzed using Cell Quest software.

Flow cytometric analysis of the spleens and tumors indicated that human CD8+ cells had dramatically expanded in the anti-TGF-β treated mice. Control mice had a median of 0% CD8+ cells in their spleens. These mice rarely had human cells in the spleens, and when human cells were present, they were human B cells. In contrast, animals receiving 125 μg anti-TGF-β had a median of 17.5% CD8+ cells in their spleens. The one treated animal that developed a B cell tumor had significant numbers of B cells in the spleen (25%), as well as significant numbers of CD8+ cells (25%). Importantly, CD8+ T cells were also expanded in the tumor of the one tumor-positive anti-TGF-β treated animal.

To determine the mechanism by which anti-TGF-β prolonged survival, additional experiments were performed with both a different antibody and using additional PBL donors. Control-treated mice had B cell tumors with very few (<5%) infiltrating CD8+ T cells. Spleens of these animals had B cell infiltration but no CD8+ T cell infiltration. In contrast, neutralization of TGF-β resulted in a dramatic expansion of human CD8+ cells in the tumors. These CD8+ cells were CD45RO and CD25+, indicating they were activated memory cells. CD45RO+, CD8+ T cells also infiltrated the spleens of these mice, but did not express CD25.

### Example 8

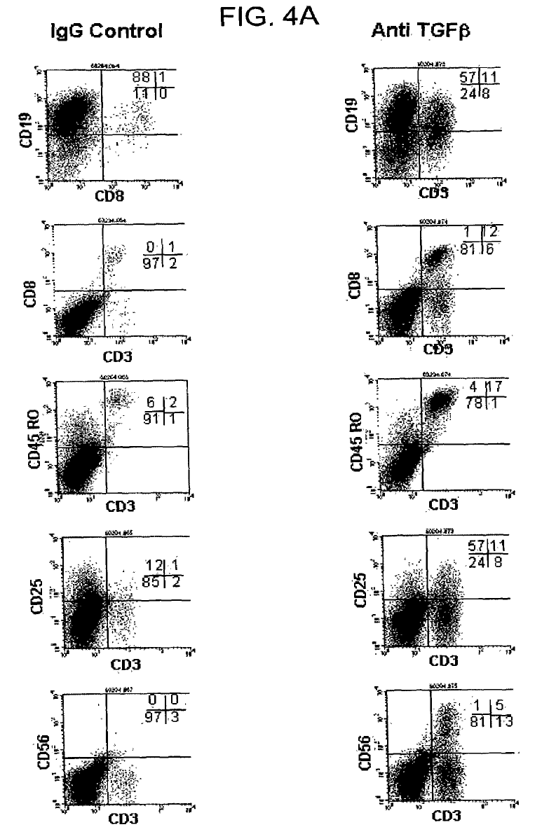

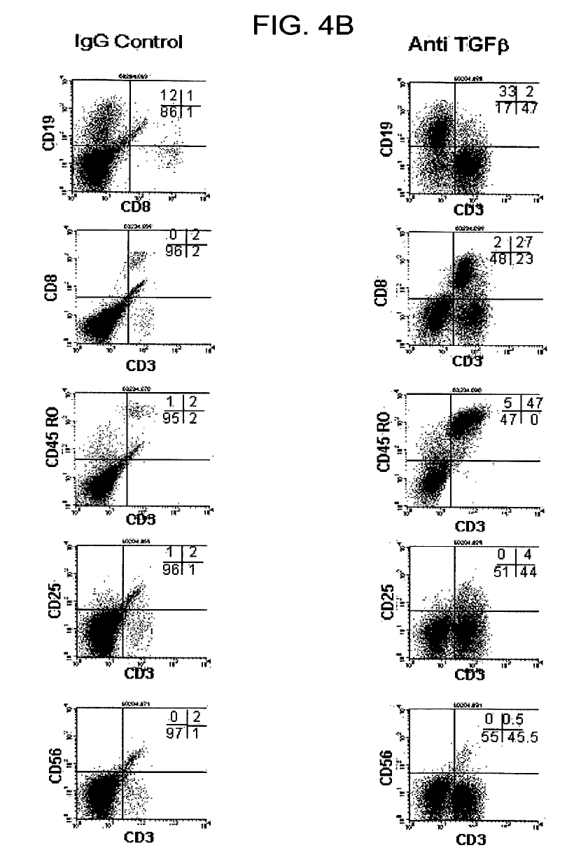

To further examine the effects of anti-TGF-β, an additional study using a third donor was performed. Hu PBL-SCID mice were injected with 100 μg anti-TGF-β antibody (A411) or mouse IgG every other day for 9 weeks (FIG. 4). Anti-TGF-β treatment effectively neutralized TGF-β in the sera of these animals (not shown). Flow cytometry was used to assess the expansion of human cells in the tumors (FIG. 4A) and spleens (FIG. 4B).

FIG. 4A shows a flow cytometric analysis of tumors in anti-TGF-β and control treated hu PBL-SCID mice. Tumors were analyzed (at harvest) by flow cytometry for the presence of human B cells and T cell expansion and activation. Data are shown from a representative animal in each group (n=5 mice per group).

FIG. 4B shows cytometric analysis of spleens from anti-TGF-β and control treated hu PBL-SCID mice. Hu PBL-SCID mice were injected with 100 μg anti-TGF-β (A411) or mouse IgG every other day for 9 weeks. At harvest, spleens were analyzed by flow cytometry for the presence of human B cells and T cell expansion and activation. Data are shown from a representative animal in each group (n=5 mice per group).

These data demonstrate that tumors from control IgG-treated mice contained human B cells and very few CD8+ T cells. Likewise, spleens from these animals contained B cells but very few if any T cells. In contrast, tumors and spleens from anti-TGF-β treated mice exhibited large numbers of CD8+ T cells. These CD8+ cells were predominantly memory cells expressing CD45RO, and in the tumors, the majority of the CD8+ cells also expressed CD25, indicating that they were activated. The majority of CD8+ cells in the spleens did not express CD25.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and biological sequences cited in this disclosure are incorporated by reference in their entirety. To the extent the material incorporated by reference contradicts or is inconsistent with the present specification, the present specification will supersede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating, preventing, or reducing the risk of occurrence of a viral-associated lymphoproliferative disorder in a mammalian subject, comprising administering a therapeutically effective amount of a TGF-β antagonist to the subject, wherein the subject has or is at risk for a viral-associated lymphoproliferative disorder.

2. The method of claim 1, wherein the viral-associated proliferative disorder is associated with a virus chosen from a herpes virus, HHV-8, cytomegalovirus, Epstein-Barr virus (EBV), C-type retrovirus, human T-lymphotropic virus type 1, and human immunodeficiency virus.

3. The method of claim 2, wherein the viral-associated lymphoproliferative disorder is an Epstein-Barr virus-associated lymphoproliferative disorder.

4. The method of claim 1, wherein the viral-associated lymphoproliferative disorder is post-transplant lymphoproliferative disorder.

5. The method of claim 1, wherein the subject wherein the subject has a low producer IFN-γ genotype.

6. The method of claim 5, wherein the subject has an adenosine at position +874 of an IFN-γ gene.

7. The method of claim 1, wherein the TGF-β antagonist is chosen from an anti-TGF-β antibody, an anti-TGF-β receptor antibody, and soluble TGF-β receptor.

8. The method of claim 7, wherein the anti-TGF-β antibody or the anti-TGF-β receptor antibody is human or humanized.

9. The method of claim 7, wherein the anti-TGF-β antibody specifically binds to TGF-β1, TGF-β2, and TGF-β3.

10. The method of claim 7, wherein the anti-TGF-β antibody specifically binds to TGF-β1 and TGF-β2.

11. The method of claim 7, wherein the antibody is 1D11 or a human or humanized derivative thereof.

12. The method of claim 7, wherein the antibody specifically binds to TGF-β1.

13. The method of claim 12, wherein the antibody is CAT192 or a derivative thereof.

14. The method of claim 4, wherein the subject is at risk due to a transplant.

15. The method of claim 14, wherein the transplant is chosen from heart, kidney, lung, liver, cornea, bone marrow, stem cell, blood vessel, and islet cell transplant.

16. The method of claim 1, wherein the subject is at risk due to immune deficiency.

17. The method of claim 1, wherein the subject is at risk due immunosuppressive therapy.

18. A method for enhancing T cell responsiveness to viral infection in a mammalian subject, comprising administering a therapeutically effective amount of a TGF-β antagonist to the subject, wherein the subject has or is at risk for a viral-associated lymphoproliferative disorder.

19. The method of claim 18, wherein the viral-associated lymphoproliferative disorder is associated with a virus chosen from a herpes virus, HHV-8, cytomegalovirus, Epstein-Barr virus (EBV), C-type retrovirus, human T-lymphotropic virus type 1, and human immunodeficiency virus.

20. The method of claim 18, wherein the viral-associated lymphoproliferative disorder is a herpes virus-associated lymphoproliferative disorder.

21. The method of claim 20, wherein the viral-associated proliferative disorder is an EBV-associated lymphoproliferative disorder.

22. The method of claim 21, wherein the EBV-associated lymphoproliferative disorder is chosen from primary CNS lymphoma, post-transplant lymphoproliferative disorder, Burkitt's lymphoma, T-cell lymphoma, X-linked lymphoproliferative disorder, Chédiak-Higashi syndrome, and Hodgkin's lymphoma.

23. The method of claim 18, wherein the viral-associated lymphoproliferative disorder is an HIV-associated lymphoproliferative disorder.

24. A method of enhancing T-cell responsiveness to a viral-associated lymphoproliferative disorder, comprising administering a therapeutically effective amount of a TGF-β antagonist to a mammalian subject in need thereof and thereby reducing aberrant cell proliferation.

25. A method of treating a viral-associated lymphoproliferative disorder associated with low IFN-γ levels, comprising administering a therapeutically effective amount of a TGF-β antagonist to a mammalian subject in need thereof.

26. A method of treating a viral-associated lymphoproliferative disorder associated with high TGF-β levels, comprising administering a therapeutically effective amount of a TGF-β antagonist to a mammalian subject in need thereof.

27. A method of identifying a candidate subject for administration of a TGF-β antagonist to treat, prevent, or reduce the risk of occurrence of a viral-associated lymphoproliferative disorder, comprising determining if a subject has a low producer IFN-γ genotype.

28. The method of claim 27, wherein the subject is homozygous for a low producer IFN-γ genotype.

29. The method of claim 27, wherein the subject is heterozygous for a low producer IFN-γ genotype.

30. The method of claim 27, wherein the subject has an adenosine at position +874 of an IFN-γ gene.

31. The method of claim 27, wherein the subject is at risk for a viral-associated lymphoproliferative disorder.

32. The method of claim 27, wherein the subject has a viral-associated lymphoproliferative disorder.

33. A method of identifying a candidate subject for administration of a TGF-β antagonist to treat, prevent, or reduce the risk of occurrence of a viral-associated lymphoproliferative disorder, comprising determining if a subject has low IFN-γ levels.

34. A method of selecting a candidate subject for administration of a TGF-β antagonist to treat a viral-associated lymphoproliferative disorder, comprising determining if the subject has a low producer IFN-γ genotype.

35. The method of claim 34, further comprising determining if the subject has an adenosine at position +874 of an IFN-γ gene.

36. A method of selecting a candidate for administration of a TGF-β antagonist to treat a viral-associated lymphoproliferative disorder, comprising determining if the subject has low IFN-γ levels.
